(19) United States
(12) Patent Application Publication
(10) Pub. No.: US 2024/0417660 A1
(43) Pub. Date: Dec. 19, 2024

(54) PLATELET EXOSOME ENRICHMENT TUBE

(71) Applicant: Pen Gang CHENG, Taichung City (TW)

(72) Inventor: Pen Gang CHENG, Taichung City (TW)

(21) Appl. No.: 18/724,199

(22) PCT Filed: Feb. 13, 2023
(86) PCT No.: PCT/CN2023/075749

§ 371 Date: Jun. 26, 2024

(30) Foreign Application Priority Data
Jun. 6, 2022 (CN) 202221391576.1

(51) Int. Cl. C12M 1/12; C12M 1/00

(52) U.S. Cl. CPC C12M 23/06; C12M 23/38

(57) ABSTRACT

A platelet exosome enrichment tube is provided. The platelet exosome enrichment tube includes a tube body, a cover body and a plurality of frosted glass beads. The tube body has a closed end, an open end and an accommodation space. The open end is opposite to the closed end, and the accommodation space is formed by connecting the closed end to the open end. The cover body detachably covers the open end of the tube body, and the frosted glass beads are disposed in the accommodation space. A surface of each of the frosted glass beads has a uniformly distributed uneven shape.

### BACKGROUND

### Technical Field

The present disclosure relates to a blood sampling device. More particularly, the present disclosure relates to a platelet exosome enrichment tube.

### Description of Related Art

Human blood mainly consists of plasma and blood cells, and the main components of blood cells are red blood cells, white blood cells and platelets. Whole blood can be prepared into a variety of ingredients, and can be effectively used in a variety of treatments stored under ideal conditions. In clinical treatments, platelets have been found to have the effect of coagulating in vitro and quickly activating endogenous growth factors, and have good effects related to promoting cell growth and repair in the human body.

With the development of modern medicine, trace blood components such as platelet rich plasma (PRP) are widely used in clinical surgery and medical cosmetology, in which blood from the subject's own body is extracted, platelet rich plasma is collected, and this is re-injected into the patient's skin or tissue surface through medical means to achieve the treatment and recovery of the body's own medical effects. Thus, rejection reactions that occur when using exogenous blood products can be avoided.

PRP uses exosomes on the surface of the platelet to secrete growth factors and anti-inflammatory factors to treat physical discomfort, which is mainly used for osteoarthritis, muscle and ligament injuries, anti-inflammation and tissue repair. In addition, PRP can also be used in medical cosmetology and dentistry. For example, its application in medical cosmetology can reduce wrinkles, and its application in dentistry can fill dental pulp, promote close contact between dental implant metal and a pulp cavity and increase growth, and thus can increase the success rate of dental implants.

However, in addition to exosomes on the surface of the platelet that can secrete growth factors and anti-inflammatory factors, there are also three isoforms of exosomes in a cytoplasm of the platelet. The conventional PRP only activates exosomes on the surface of the platelet, so only the growth factors on the surface of the platelet are activated and released, and the other three isomers of exosomes inside the cell membrane of the platelet are not utilized. Therefore, it is necessary to develop a clinical medical device that can break the cell membrane of the platelet, and then use the exosome activation of the three isomers inside the cell membrane of the platelet to release growth factors and anti-inflammatory factors to improve the efficacy and scope of clinical application.

### SUMMARY

One embodiment of the present disclosure provides a platelet exosome enrichment tube, which includes a tube body, a cover body and a plurality of frosted glass beads. The tube body has a closed end, an open end, and an accommodation space, wherein the open end is opposite to the closed end, and the accommodation space is formed by connecting the closed end to the open end. The cover body detachably covers the open end of the tube body to open and close the accommodation space. The frosted glass beads are disposed in the accommodation space, wherein a surface of each of the plurality of frosted glass beads has a uniformly distributed uneven shape.

### BRIEF DESCRIPTION OF THE DRAWINGS

The present disclosure can be more fully understood by reading the following detailed description of the embodiment, with reference made to the accompanying drawings as follows:

FIG. 1 is an exploded view of a platelet exosome enrichment tube according to one embodiment of the present disclosure;

FIG. 2 is an exploded view of a platelet exosome enrichment tube according to another embodiment of the present disclosure;

FIG. 3 is a perspective view of a platelet exosome enrichment tube according to still another embodiment of the present disclosure;

FIG. 4 is an assembled cross-sectional view of the platelet exosome enrichment tube of FIG. 3;

FIG. 5 is a perspective view of a platelet exosome enrichment tube according to yet another embodiment of the present disclosure; and

FIG. 6 is an assembled cross-sectional view of the platelet exosome enrichment tube of FIG. 5.

### DETAILED DESCRIPTION

The embodiment will be described with reference to the drawings. For clarity, some practical details will be described below. However, it should be noted that the present disclosure should not be limited by the practical details, that is, in some embodiment, the practical details are unnecessary. In addition, for simplifying the drawings, some conventional structures and elements will be simply illustrated, and repeated elements may be represented by the same reference numerals.

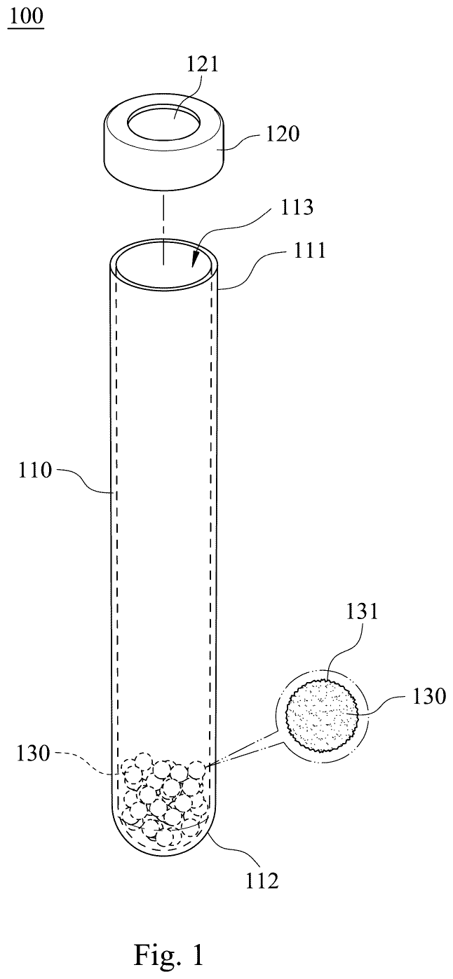

Reference is made to FIG. 1, which is an exploded view of a platelet exosome enrichment tube 100 according to one embodiment of the present disclosure. The platelet exosome enrichment tube 100 includes a tube body 110, a cover body 120 and a plurality of frosted glass beads 130.

The tube body 110 has a closed end 112, an open end 111, and an accommodation space 113, wherein the open end 111 is opposite to the closed end 112, and the accommodation space 113 is formed by connecting the closed end 112 to the open end 111. The closed end 112 of the tube body 110 can be arc-shaped, so that the platelet exosome enrichment tube 100 can concentrate a blood sample at the tube bottom when in use. A material of the tube body 110 can be a glass or a plastic; preferably, the material can be transparent or translucent plastic or glass, so that the blood layering can be clearly seen when the platelet exosome enrichment tube 100 is used. In addition, the outer wall surface of the tube body 110 can have a plurality of scale marks, so that the internal volume can be directly identified when in use. More preferably, an inner diameter of the tube body 110 can be 10 mm to 20 mm, a length of the tube body 110 can be 100 mm to 150 mm, and the tube body 110 can accommodate a blood sample of 10 mL to 15 mL, but the present disclosure is not limited thereto.

The cover body 120 detachably covers the open end 111 of the tube body 110 to open and close the accommodation space 113. The cover body 120 can be a spiral tube cap, a plastic cap, a safety cap or a rubber cap. When the cover body 120 is made of an inelastic material such as the spiral tube cap, the plastic cap or the safety cap, the open end 111 of the tube body 110 has an external thread, an inner surface of the cover body 120 has an internal thread, and the tube body 110 and the cover body 120 are tightly connected through the external thread and the internal thread. When the cover body 120 is made of an elastic material such as the rubber cap, the cover body 120 can be directly sleeved on the open end 111 of the tube body 110. The cover body 120 can include an injection port 121 for injecting and/or extracting a blood sample with a blood sampling needle. The blood sampling needle can be hard-connected or soft-connected.

The frosted glass beads 130 are disposed in the accommodation space 113, wherein a surface 131 of each of the frosted glass beads 130 has a uniformly distributed uneven shape. The uniformly distributed uneven shape of the surface 131 can be formed by a chemical treatment method or a physical treatment method. The chemical treatment method is, for example, a frosting treatment method, in which the glass beads are immersed in a mixed solution of hydrogen fluoride and ammonium fluoride, and the immersed surface is chemically surface treated to obtain the frosted glass beads 130. The physical treatment method includes a sandblasting treatment using pressurized air to blow crystalline silicon dioxide powder and silicon carbide powder onto the surface of the glass beads to obtain the frosted glass beads 130; or rubbing the glass beads with a brush moistened with water and attached with crystalline silicon dioxide powder and silicon carbide powder to obtain the frosted glass beads 130. A surface roughness (RMS) of each of the frosted glass beads 130 can be 0.01 μm to 0.5 μm, and an average particle size of each of the frosted glass beads 130 can be 2 mm to 10 mm. More preferably, 20 to 50 frosted glass beads 130 can be disposed in the accommodation space 113 of the tube body 110, but the present disclosure is not limited thereto. Reference is made to the following Table 1 to Table 3, Table 1 shows size-related properties of the frosted glass bead 130, Table 2 shows chemical properties of the frosted glass bead 130, and Table 3 shows physical properties of the frosted glass bead 130.

TABLE 1
Size-related properties of frosted glass bead 130

| Average | Compressive | Amount of frosted | Contact | Weight |
|---|---|---|---|---|
| particle size | strength | glass bead in 1 kg | surface in 1 kg | of 1 L |
| (mm) | (N) | (ks/kg) | (cm²) | (kg/1 L) |
| 2 | 950 | 107527 | 13505.5 | 1.402 |
| 3 | 1650 | 31746 | 8970.4 | 1.407 |
| 4 | 2350 | 13387 | 6725.5 | 1.408 |
| 5 | 2700 | 6849 | 5376.6 | 1.409 |
| 6 | 3700 | 3965 | 4481.8 | 1.408 |
| 7 | 3950 | 2497 | 3841.4 | 1.409 |
| 8 | 5350 | 1673 | 3362.8 | 1.409 |
| 9 | 5850 | 1656 | 4211.1 | 1.409 |
| 10 | 6350 | 1207 | 3790.0 | 1.408 |

TABLE 2
Chemical properties of frosted glass bead 130

| Composition | Contents | Tolerance of Contents |
|---|---|---|
| Silicon dioxide (SiCO₂) | 80.3% | +1.0 |
| Boron trioxide (B₂O₃) | 13.0% | +0.5 |
| Sodium oxide (Na₂O) + Potassium | 4.3% | +0.5 |
| Oxide (K₂O) | | |
| Aluminum oxide (Al₂O₃) | 2.4% | +0.5 |

TABLE 3
Physical properties of frosted glass bead 130

| Physical properties | Numerical value |
|---|---|
| Density | 2.230 kg/dm³ |
| Coefficient of thermal | (3.3 ± 0.4) × 10⁻⁶K⁻¹ |
| expansion | |
| Littleton point | TL = 787 ± 10° C. |
| Bulk density | 1.409 kg/dm³ |
| Mohs hardness | 7 |
| Young's modulus | 64 Gpa |
| Hydrolytic class | HGB 1 |
| Acidic resistance - | S1 |
| DIN 12116 | |
| Alkaline resistance - | A1 |
| DIN ISO 695 | |
| Refractive index | 1.47 |

The platelet exosome enrichment tube 100 can use physical principle to break the cell membrane of the platelet when preparing the platelet exosome enrichment solution by using the frosted glass beads 130 with uniformly distributed uneven shape on the surface 131. The other three isomers of exosomes in the cytoplasm of the platelets can also be activated and released, thereby obtaining a platelet exosome enrichment solution containing multiple anti-inflammatory factors and growth factors. Moreover, the concentration of various growth factors in the obtained platelet exosome enrichment solution is several times higher than that of the conventional platelet rich plasma, making the subsequent clinical treatment more effective.

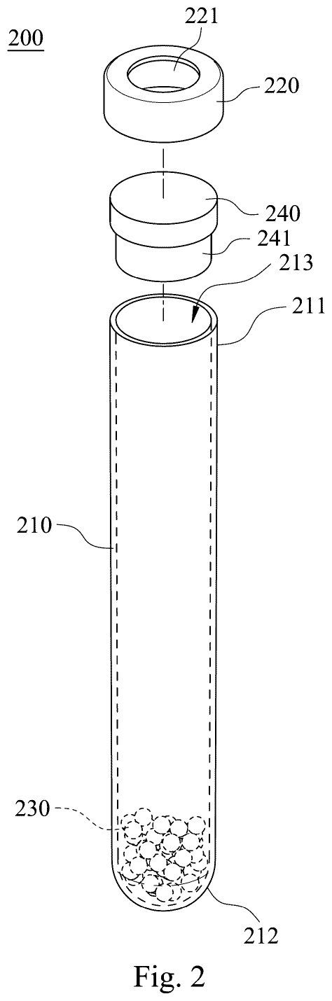

Reference is made to FIG. 2, which is an exploded view of a platelet exosome enrichment tube 200 according to another embodiment of the present disclosure. The platelet exosome enrichment tube 200 includes a tube body 210, a cover body 220, a plurality of frosted glass beads 230 and a rubber stopper 240.

The tube body 210 has a closed end 212, an open end 211, and an accommodation space 213, wherein the open end 211 is opposite to the closed end 212, and the accommodation space 213 is formed by connecting the closed end 212 to the open end 211. Other technical details of the tube body 210 are the same as those of the tube body 110, and will not be described again.

The cover body 220 detachably covers the open end 211 of the tube body 210 to open and close the accommodation space 213. The cover body 220 can include an injection port 221 for injecting and/or extracting a blood sample with a blood sampling needle. Other technical details of the cover body 220 are the same as those of the cover body 120, and will not be described again.

The frosted glass beads 230 are disposed in the accommodation space 213, wherein a surface (not shown) of each of the frosted glass beads 230 has a uniformly distributed uneven shape. Other technical details of the frosted glass beads 230 are the same as those of the frosted glass beads 130, and will not be described again.

The platelet exosome enrichment tube 200 can further include the rubber stopper 240. The rubber stopper 240 is disposed between the tube body 210 and the cover body 220 to seal the accommodation space 213. An outermost diameter of the rubber stopper 240 can be the same as an outer diameter of the tube body 210. In addition, the rubber stopper 240 can have a protruding portion 241 at one end close to the tube body 210, and an outer diameter of the protruding portion 241 is the same as an inner diameter of the tube body 210. The rubber stopper 240 can be inserted into the open end 211 of the tube body 210 through the protruding portion 241, so that the rubber stopper 240 is disposed at the open end 211 of the tube body 210, and then the cover body 220 is covered outside the rubber stopper 240. The rubber stopper 240 can be made of butyl rubber, so that a blood sampling needle can pierce the rubber stopper 240 through the injection port 221 of the cover body 220 and enter the accommodation space 213 of the tube body 210.

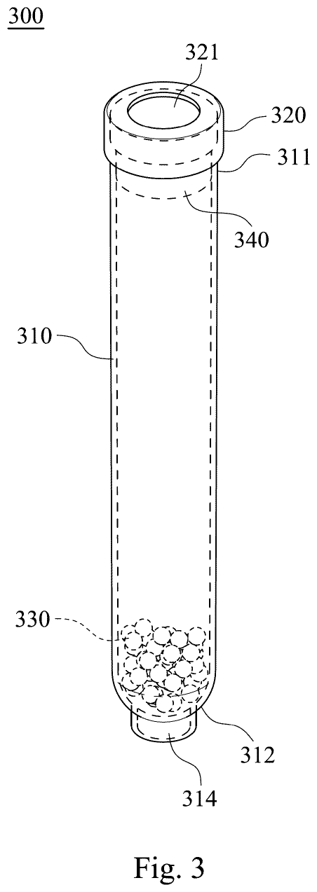

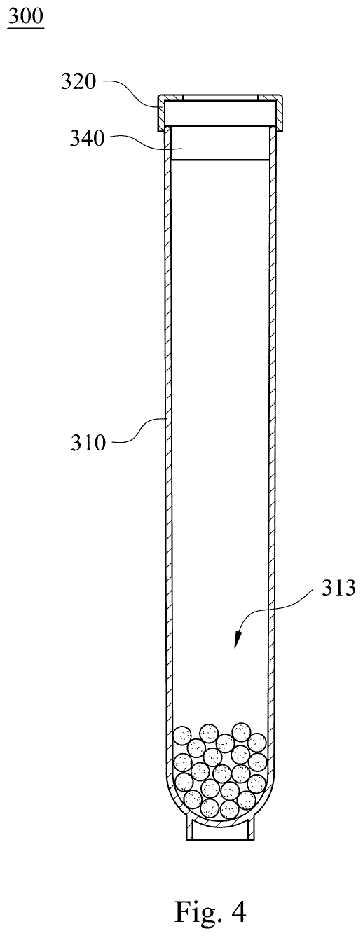

Reference is made to FIG. 3 and FIG. 4. FIG. 3 is a perspective view of a platelet exosome enrichment tube 300 according to still another embodiment of the present disclosure, and FIG. 4 is an assembled cross-sectional view of the platelet exosome enrichment tube 300 of FIG. 3. The platelet exosome enrichment tube 300 includes a tube body 310, a cover body 320, a plurality of frosted glass beads 330 and a rubber stopper 340.

The tube body 310 has a closed end 312, an open end 311, an accommodation space 313 and a standing portion 314, wherein the open end 311 is opposite to the closed end 312, and the accommodation space 313 is formed by connecting the closed end 312 to the open end 311. The closed end 312 of the tube body 310 can be arc-shaped, so that the platelet exosome enrichment tube 300 can concentrate a blood sample at the tube bottom when in use. The tube body 310 of the platelet exosome enrichment tube 300 can further include the standing portion 314 connected to the closed end 312, and one end of the standing portion 314 away from the closed end 312 is a flat bottom edge. As shown in FIG. 4, since one end of the standing portion 314 is the flat bottom edge, the platelet exosome enrichment tube 300 can stand upright on the desktop which is used. There is no need to use a test tube rack to place or store the platelet exosome enrichment tube 300, thereby increasing the convenience of use.

The cover body 320 detachably covers the open end 311 of the tube body 310 to open and close the accommodation space 313. The cover body 320 can include an injection port 321 for injecting and/or extracting a blood sample with a blood sampling needle. Other technical details of the cover body 320 are the same as those of the cover body 120, and will not be described again.

The frosted glass beads 330 are disposed in the accommodation space 313, wherein a surface (not shown) of each of the frosted glass beads 330 has a uniformly distributed uneven shape. Other technical details of the frosted glass beads 330 are the same as those of the frosted glass beads 130, and will not be described again.

The rubber stopper 340 is disposed between the tube body 310 and the cover body 320 to seal the accommodation space 313. Other technical details of the rubber stopper 340 are the same as those of the rubber stopper 240, and will not be described again.

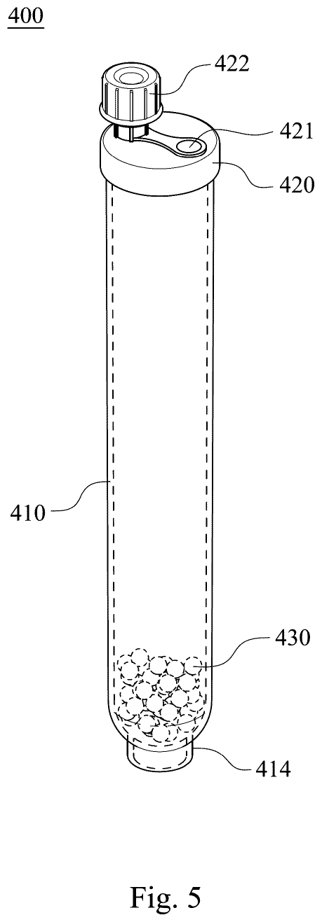

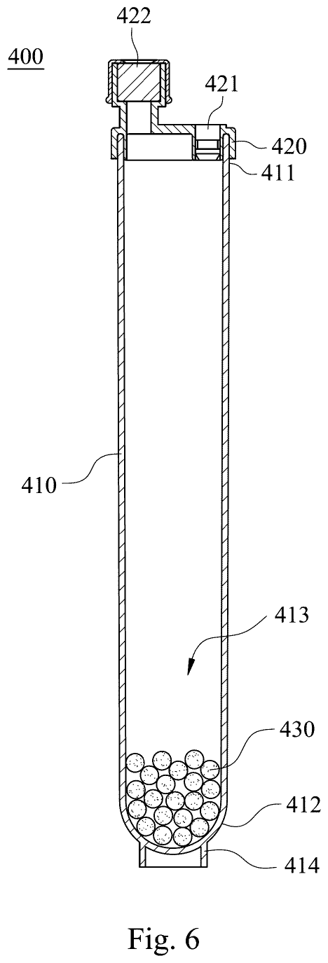

Reference is made to FIG. 5 and FIG. 6. FIG. 5 is a perspective view of a platelet exosome enrichment tube 400 according to yet another embodiment of the present disclosure, and FIG. 6 is an assembled cross-sectional view of the platelet exosome enrichment tube 400 of FIG. 5. The platelet exosome enrichment tube 400 includes a tube body 410, a cover body 420 and a plurality of frosted glass beads 430.

The tube body 410 has a closed end 412, an open end 411, an accommodation space 413 and a standing portion 414, wherein the open end 411 is opposite to the closed end 412, and the accommodation space 413 is formed by connecting the closed end 412 to the open end 411. The standing portion 414 is connected to the closed end 412, and one end of the standing portion 414 away from the closed end 412 is a flat bottom edge, so that the platelet exosome enrichment tube 400 can stand upright on the desktop which is used. Other technical details of the tube body 410 are the same as those of the tube body 110, and will not be described again.

The cover body 420 detachably covers the open end 411 of the tube body 410 to open and close the accommodation space 413. The cover body 420 can include an injection port 421 for injecting and/or extracting a blood sample with a blood sampling needle. The cover body 420 of the platelet exosome enrichment tube 400 can further include a connecting portion 422 protruding from an upper surface of the cover body 420 for connecting with a negative pressure suction device to increase the applicable range of the platelet exosome enrichment tube 400. Other technical details of the cover body 420 are the same as those of the cover body 120, and will not be described again.

The frosted glass beads 430 are disposed in the accommodation space 413, wherein a surface (not shown) of each of the frosted glass beads 430 has a uniformly distributed uneven shape. Other technical details of the frosted glass beads 430 are the same as those of the frosted glass beads 130, and will not be described again.

### Test Example

Platelets can secrete a variety of growth factors and anti-inflammatory factors, and have functions such as promoting cell movement, differentiation, division, matrix proliferation, angiogenesis, and balance. They include important growth factors such as platelet-derived growth factor (PDGF), transforming growth factor α (TGFα), transforming growth factor β (TGFβ), insulin-like growth factor 1 (IGF-1), vascular endothelial growth factor (VEGF), and interleukin 1 receptor antagonist (IL1-Ra). PDGF has functions such as attracting stem cells, promoting stem cell division, extracellular matrix secretion, and stimulating osteogenesis. TGFα can promote the secretion of extracellular matrix and promote the maturation of mother cells. TGFβ can help improve allergic constitution and regulate the normal development of the immune system. The TGFβ superfamily includes TGFβ1, TGFβ2 and TGFβ3. IGF-1 can promote fracture healing and the growth of skin, muscles, blood vessels, tendons and ligaments. VEGF can promote angiogenesis and inhibit osteoclasts. IL1-Ra can inhibit the inflammatory factor interleukin 1 and is an important anti-inflammatory factor.

In order to test whether the platelet exosome enrichment tube of the present disclosure can prepare a platelet exosome enrichment solution containing multiple anti-inflammatory factors and growth factors, in this test example, a blood sample extracted from a subject was injected into the platelet exosome enrichment tube of the present disclosure with a blood sampling needle, and then placed in an incubator at 37° C. to stabilize platelet activity. After 1-4 hours, the blood sample was taken out from the incubator, placed in the inner sleeve of a flywheel centrifuge, and centrifuged at 4000 rpm/min for 5 minutes. At this time, the blood sample was separated into a platelet exosome enrichment liquid layer and a blood cell layer. The platelet exosome enrichment tube was taken out after centrifugation, and a long needle was inserted into the tube body from the injection port of the cover body to extract the platelet exosome enrichment liquid in the upper layer. That is, a prepared platelet exosome enrichment solution was obtained, which can be subsequently injected into the desired site of the subject. The platelet exosome enrichment tube used can be the platelet exosome enrichment tube shown in FIG. 1, FIG. 2, FIG. 3 or FIG. 5.

In the experiment, the concentrations of various growth factors and cytokines of the platelet exosome enrichment solution (hereinafter referred to as “PRPII”) prepared by the platelet exosome enrichment tube of the present disclosure was further compared with that in the platelet rich plasma (hereinafter referred to as “PRP”) prepared by the conventional method. Reference is made to Table 4 and Table 5. Table 4 shows the fold increase in the concentration of growth factors required for clinical application in various tissue repairs in PRPII, and the values are based on PRP. Table 5 shows the concentration of each cytokine in PRPII and PRP. The number in parentheses after PRPII represents the stabilizing time (hours) in the incubator, and the concentration unit of each cytokine is pg/mL.

TABLE 4
The fold increase in the concentration of growth factors required
for clinical application in various tissue repairs in PRPII

| Growth factor | PDGF | TNFα | TGFβ | IGF-1 | VEGF |
|---|---|---|---|---|---|
| Fold of increase | 5.4 | 1.5 | 2.2 | 1.2 | 3.1 |
| Fracture healing | ◯ | — | ◯ | ◯ | ◯ |
| Skin repair | ◯ | ◯ | ◯ | ◯ | ◯ |
| Nerve repair | ◯ | — | — | — | — |
| Blood vessel growth | — | — | — | ◯ | ◯ |
| Muscle growth | — | — | — | ◯ | — |
| Tendon and ligament | ◯ | — | ◯ | ◯ | ◯ |

TABLE 5
The concentration of each cytokine in PRPII and PRP

| Sample | IL1-Ra | PDGF | TGFβ1 | VEGF | IGF-1 | TNFα |
|---|---|---|---|---|---|---|
| PRP | 329.9 | 2305.4 | 12701.7 | 97.8 | 39737.5 | 5.38 |
| PRPII(1) | 431.6 | 12148.4 | 31841.1 | 293.6 | 47254.3 | 8.23 |
| PRPII(2) | 629.2 | 12092.1 | 30173.8 | 306.8 | 45840.6 | 8.30 |
| PRPII(3) | 1013.7 | 11184.4 | 29382.4 | 306.6 | 47127.0 | 11.14 |
| PRPII(4) | 1497.4 | 11627.8 | 30977.1 | 308.9 | 48760.4 | 10.18 |

The results in Table 4 show that the concentrations of growth factors required for tissue repair in PRPII prepared from the platelet exosome enrichment tube of the present disclosure are significantly increased compared to conventional PRP. Furthermore, the results in Table 5 show that the concentrations of growth factors are related to the stabilizing time of blood samples at 37° C. The optimal stabilizing time is 3-4 hours to obtain the best results. The concentration of anti-inflammatory factor IL1-Ra contained in PRPII is 4.15 times that of PRP, and the concentration of growth factor PDGF contained in PRPII is 5.4 times that of PRP. Although the concentration of tumor necrosis factor α (TNFα) contained in PRPII is slightly higher than that of PRP, it does not increase significantly due to the longer culture time, indicating that the platelet exosomes enrichment tube of the present disclosure can indeed prepare PRPII containing high concentrations of various growth factors.

In the experiment, PRPII prepared from the platelet exosome enrichment tube of the present disclosure was used in clinical trials to further verify the effect of PRPII on reducing the pain index of patients with degenerative arthritis after injection. The subjects in the trial were 30 patients with stage III degenerative arthritis (VAS pain scale: 8-10). The above subjects received the PRPII treatment once every 2 weeks, with a total of 5 treatments. The experiment also included subjects who received the PRP treatment as a control, and the strategy of the PRP treatment was the same as that of the PRPII treatment. Reference is made to Table 6 and Table 7, Table 6 shows the percentage of subjects in which an improvement in pain was experienced, and Table 7 shows pain scale results of subjects after the course of treatment.

TABLE 6
Percentage of pain improved of subject

| course | 1^{st} | 2^{nd} | 3^{rd} | 4^{th} | 5^{th} |
|---|---|---|---|---|---|
| PRP | 10% | 15% | 30% | 40% | 60% |
| PRPII | 40% | 60% | 70% | 80% | 100% |

TABLE 7
Pain scale result of subjects after the course of treatment

| Pain scale | Reduced to 2-4 | Reduced to 5-6 | Maintain at 8-10 |
|---|---|---|---|
| PRP | 15 (50%) | 5 (17%) | 10 (33%) |
| PRPII | 25 (80%) | 5 (20%) | N/A |

The results in Table 6 and Table 7 show that all subjects who received PRPII treatment experienced improvement in pain after the course of treatment, wherein 80% of the subjects had their pain scale reduced from 8-10 to 2-4. The results indicate that PRPII prepared from the platelet exosome enrichment tube of the present disclosure can significantly reduce the pain of patients with degenerative arthritis.

In addition to the clinical application of injection treatment of degenerative arthritis and tendon injuries, PRPII prepared from the platelet exosome enrichment tube of the present disclosure can also be used in a variety of clinical treatments, such as dental implant treatments, medical aesthetic treatments, alopecia treatments, orthopedic treatments, trauma treatments, ophthalmology treatments, obstetrics and gynecology treatments, etc. When used for dental implant treatments, PRPII is mixed with hyaluronic acid to form a semi-solid filling and fill in a dental implant site, which can promote the close connection between the dental implant and bone and increase the chance of successful dental implantation. When used for medical aesthetic treatments, PRPII can be injected subcutaneously to promote collagen regeneration and improve skin texture. PRPII can also be used simultaneously with facial injection of hyaluronic acid to multiply the effect. When used for alopecia treatments, PRPII can be injected into the scalp to regenerate hair. When used for orthopedic treatments, PRPII is directly injected into the fracture defect after surgery to promote bone healing, or PRPII is injected into the bone cavity to promote growth. When used for trauma treatments, PRPII can be combined with artificial skin to cover diabetic feet and other chronic ulcerative wounds that are difficult to heal, such as bedsores or burns, to accelerate healing. When used for ophthalmic treatments, PRPII can be made into eye drops to treat dry eye syndrome and chronic corneal ulcers or corneal dystrophy. When used for obstetrics and gynecology treatments, PRPII can be injected into the uterus through the cervix to thicken the endometrium, thereby improving infertility caused by insufficient endometrium hypertrophy and increasing the success rate of pregnancy.

In summary, the platelet exosome enrichment tube of the present disclosure includes the frosted glass beads with uniformly distributed uneven shape on the surface. When preparing a platelet exosome enrichment solution, the cell membrane of the platelet can be broken by physical principle, so that in addition to exosomes on the surface of the platelet, the other three isomers of exosomes in the cytoplasm can also be activated and released, thereby obtaining a platelet exosome enrichment solution containing multiple anti-inflammatory factors and growth factors. The concentrations of various growth factors in the obtained platelet exosome enrichment solution are several times higher than that of the conventional platelet rich plasma, so the clinical therapeutic effects in subsequent applications are better and can be used in a variety of clinical treatments, which has great clinical application prospects.

Although the present disclosure has been described in the above by way of embodiments, it is not intended to limit the present disclosure. Persons having ordinary skill in the art can make various changes and modifications without departing from the spirit and scope of the present disclosure. Therefore, the scope of protection of the present disclosure should be defined by the scope of the following claims.

1. A platelet exosome enrichment tube, comprising:
a tube body having a closed end, an open end, and an accommodation space, wherein the open end is opposite to the closed end, and the accommodation space is formed by connecting the closed end to the open end;
a cover body detachably covering the open end of the tube body to open and close the accommodation space; and
a plurality of frosted glass beads disposed in the accommodation space, wherein a surface of each of the plurality of frosted glass beads has a uniformly distributed uneven shape.

2. The platelet exosome enrichment tube of claim 1, wherein the closed end of the tube body is arc-shaped.

3. The platelet exosome enrichment tube of claim 2, wherein the tube body further comprises a standing portion connected to the closed end, and one end of the standing portion away from the closed end is a flat bottom edge.

4. The platelet exosome enrichment tube of claim 1, wherein a material of the tube body is a glass or a plastic.

5. The platelet exosome enrichment tube of claim 1, wherein the cover body is a spiral tube cap, a plastic cap, a safety cap or a rubber cap.

6. The platelet exosome enrichment tube of claim 1, wherein the open end of the tube body has an external thread, an inner surface of the cover body has an internal thread, and the tube body and the cover body are tightly connected through the external thread and the internal thread.

7. The platelet exosome enrichment tube of claim 1, wherein the cover body comprises an injection port for injecting and/or extracting a blood sample with a blood sampling needle.

8. The platelet exosome enrichment tube of claim 7, wherein the cover body further comprises a connecting portion protruding from an upper surface of the cover body for connecting with a negative pressure suction device.

9. The platelet exosome enrichment tube of claim 1, further comprising a rubber stopper, wherein the rubber stopper is disposed between the tube body and the cover body to seal the accommodation space.

10. The platelet exosome enrichment tube of claim 1, wherein a surface roughness (RMS) of each of the plurality of frosted glass beads is 0.01 μm to 0.5 μm.

11. The platelet exosome enrichment tube of claim 1, wherein an average particle size of each of the plurality of frosted glass beads is 2 mm to 10 mm.
